(19) United States
(12) Patent Application Publication
(10) Pub. No.: US 2009/0004077 A1
(43) Pub. Date: Jan. 1, 2009

(54) Apparatus and Method for Preventing Haze Growth on a Surface of a Substrate

(76) Inventors: Larry E. Frisa, Round Rock, TX (US); Ethan M. Frye, Westfield, IL (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 12/279,069

(22) PCT Filed: Feb. 12, 2007
(86) PCT No.: PCT/US07/61973

§ 371 Date: Aug. 12, 2008

Related U.S. Application Data
(60) Provisional application No. 60/772,695, filed on Feb. 13, 2006.

(51) Int. Cl. A61L 2/20

(52) U.S. Cl. 422/300; 422/292

(57) ABSTRACT

An apparatus and method for preventing haze growth on a surface of a substrate are disclosed. The apparatus includes a container operable to store a substrate and a gas source coupled to the container. The gas source is operable to dispense a gas into the container in order to prevent a haze from growing on a surface of the substrate.

### CROSS REFERENCE TO RELATED APPLICATION

This application is a US National Stage Application of International Application of No. PCT/US2007/061973 filed Feb. 12, 2007, which designates the United States and claims the benefit under 35 U.S.C. § 119(e) of U.S. Provisional Application No. 60/772,695, filed Feb. 13, 2006. The contents of these applications are incorporated herein in their entirety by this reference.

### TECHNICAL FIELD

This disclosure relates in general to semiconductor manufacturing and, more particularly, to an apparatus and method for preventing haze growth on a surface of a substrate.

### BACKGROUND OF THE DISCLOSURE

As semiconductor device manufacturers continue to produce smaller devices, the requirements for photomasks used in the fabrication of these devices continue to tighten. Photomasks, also known as reticles or masks, typically consist of substrates (e.g., high-purity quartz or glass) that have an absorber layer (e.g., chrome or MoSi) formed on the substrate. The absorber layer includes a pattern representing a circuit image that may be transferred onto semiconductor wafers in a lithography system. As feature sizes of semiconductor devices decrease, the corresponding circuit images on the photomask also become smaller and more complex. Consequently, the quality of the mask has become one of the most crucial elements in establishing a robust and reliable semiconductor fabrication process.

Characteristics of the photomask that define its quality include the flatness of the substrate, the dimensions of the features formed by the opaque layer and the transmission properties of the substrate and the absorber layer. These characteristics may be altered by various procedures during the manufacturing process, which may reduce the quality of the photomask.

Contamination growth on optical surfaces, such as photomask and pellicle film surfaces, has become an increasing issue as the industry has adopted lower wavelengths in order to increase lithographic resolution and print ever decreasing geometries. The mask patterning process, cleaning processes, pellicle film materials and the storage environment all may introduce additional opportunities for contribution of contaminants that may contribute to or enable the growth of progressive growth defects.

In a cleaning process, for example, a chemical residue generated by the cleaning agents may remain on the exposed surfaces. If the substrate is held in a storage container for a long period of time the residue generated during the cleaning process (e.g., sulfates and/or ammonia) may react with moisture trapped in the container and cause a haze to grow on the exposed surfaces. Specifically, the haze may be caused by absorptions of sulfuric acid (H₂SO₄), ammonium hydroxide (NH₄OH) and ammonium sulfate (NH₄SO₄). The haze can alter the transmission properties of the substrate and/or cause defects on the substrate. If the transmission properties of a photomask substrate are altered, the pattern from the photomask may not be accurately transferred to a semiconductor wafer, thus causing defects or errors in the microelectronic devices formed on the wafer.

### SUMMARY OF THE DISCLOSURE

In accordance with the present disclosure, the disadvantages and problems associated with preventing haze grown on a surface of a substrate have been reduced or eliminated. In a particular embodiment, gas may be dispensed into a storage container holding a substrate such that the gas prevents haze from growing on a surface of the substrate.

In accordance with one embodiment of the present disclosure, an apparatus for preventing haze growth on a surface of a substrate includes a container operable to store a substrate and a gas source coupled to the container. The gas source is operable to dispense a gas into the container in order to prevent a haze from growing on a surface of the substrate.

In accordance with another embodiment of the present disclosure, a method for preventing haze growth on a surface of a substrate includes providing a container operable to hold a substrate and enclose the substrate within the container and a gas source coupled to the container. The gas source dispenses a gas into the container in order to prevent a haze from growing on a surface of the substrate.

### BRIEF DESCRIPTION OF THE DRAWINGS

A more complete understanding of the present embodiments and advantages thereof may be acquired by referring to the following description taken in conjunction with the accompanying drawings, in which like reference numbers indicate like features, and wherein:

FIG. 1 illustrates a cross-sectional view of a photomask assembly according to teachings of the present disclosure;

FIGS. 2A and 2B respectively illustrate a top view and a side view of a storage container for holding a substrate according to teachings of the present disclosure; and

FIGS. 3A through 3C illustrate graphs of contaminant growth on a surface of a substrate over time when exposed to a gas according to teachings of the present disclosure.

### DETAILED DESCRIPTION OF THE DISCLOSURE

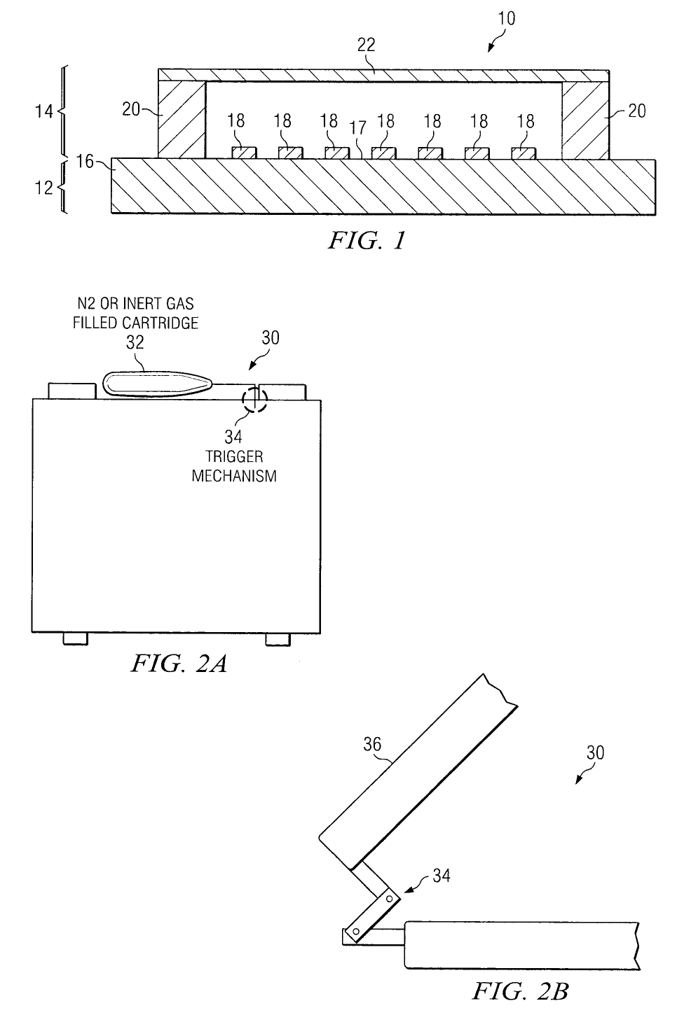

Preferred embodiments of the present disclosure and their advantages are best understood by reference to FIGS. 1 through 3, where like numbers are used to indicate like and corresponding parts.

A photomask is a crucial component of a lithography system because it serves as the template that images a complex geometry, such as an integrated circuit (IC), on a wafer. During manufacturing, different gases and/or chemicals may be introduced proximate to the surface of the photomask. Although each photomask may be cleaned at least one time, some residue may remain on the surface. This residue may react with other contaminants generated when the photomask is used in a lithography process and/or any contaminants present in a storage container used to store the photomask. These reactions may cause a haze to grow on the photomask, which can alter the transmission properties of the photomask and damage the patterned layer on the photomask.

The present disclosure provides an apparatus that prevents haze growth from occurring on a surface of a substrate. A substrate, such as a photomask, pellicle film or semiconductor wafer, may be placed in a storage container for transport to another manufacturing facility or for storage until the substrate is needed in a manufacturing process. The storage container may include a gas source that discharges a gas into the container. Once the substrate has been placed in the storage container, a lid attached to the container may be closed to seal the substrate in the storage container. The motion of the lid being closed may trigger the gas source to release a gas into the container. This gas may prevent a haze from growing on the surface of the substrate while the substrate is sealed in the storage container.

FIG. 1 illustrates a cross-sectional view of an example photomask assembly 10. Photomask assembly 10 includes pellicle assembly 14 mounted on photomask 12. Substrate 16, and patterned layer 18 form photomask 12, otherwise known as a mask or reticle, that may have a variety of sizes and shapes, including but not limited to round, rectangular, or square. Photomask 12 may also be any variety of photomask types, including, but not limited to, a one-time master, a five-inch reticle, a six-inch reticle, a nine-inch reticle or any other appropriately sized reticle that may be used to project an image of a circuit pattern onto a semiconductor wafer. Photomask 12 may further be a binary mask, a phase shift mask (PSM) (e.g., an alternating aperture phase shift mask, also known as a Levenson type mask), an optical proximity correction (OPC) mask or any other type of mask suitable for use in a lithography system. In other embodiments, photomask 12 may be a step and flash imprint lithography (SFIL) template used to form an imprint of a pattern in a polymerizable fluid composition that solidifies to form a device on a wafer. The template may be a semi-transparent material, and the polymerizable fluid may be solidified by exposure to a radiation source in order to form the device on the wafer.

Photomask 12 includes patterned layer 18 formed on top surface 17 of substrate 16 that, when exposed to electromagnetic energy in a lithography system, projects a pattern onto a surface of a semiconductor wafer (not expressly shown). Substrate 16 may be a transparent material such as quartz, synthetic quartz, fused silica, magnesium fluoride (MgF₂), calcium fluoride (CaF₂), or any other suitable material that transmits at least seventy-five percent (75%) of incident light having a wavelength between approximately 10 nanometers (nm) and approximately 450 nm. In an alternative embodiment, substrate 16 may be a reflective material such as silicon or any other suitable material that reflects greater than approximately fifty percent (50%) of incident light having a wavelength between approximately 10 nm and 450 nm.

Patterned layer 18 may be a metal and/or metallic compound such as chromium, chromium nitride, a metallic oxy-carbo-nitride (e.g., MOₓC_{y}N_{z}, where M is selected from the group consisting of chromium, cobalt, iron, zinc, molybdenum, niobium, tantalum, titanium, tungsten, aluminum, magnesium, and silicon), or any other suitable material that absorbs electromagnetic energy with wavelengths in the ultraviolet (UV) range, deep ultraviolet (DUV) range, vacuum ultraviolet (VUV) range and extreme ultraviolet range (EUV). In an alternative embodiment, patterned layer 18 may be a partially transmissive material, such as molybdenum silicide (MoSi), which has a transmissivity of approximately one percent (1%) to approximately thirty percent (30%) in the UV, DUV, VUV and EUV ranges.

Frame 20 and pellicle film 22 may form pellicle assembly 14. Frame 20 is typically formed of anodized aluminum, although it could alternatively be formed of stainless steel, plastic or other suitable materials that do not degrade or outgas when exposed to electromagnetic energy within a lithography system. Pellicle film 22 may be a thin film membrane formed of a material such as nitrocellulose, cellulose acetate, an amorphous fluoropolymer, such as TEFLON® AF manufactured by E. I. du Pont de Nemours and Company or CYTOP® manufactured by Asahi Glass, or another suitable film that is transparent to wavelengths in the UV, DUV, EUV and/or VUV ranges. Pellicle film 22 may be prepared by a conventional technique such as spin casting.

Pellicle film 22 protects photomask 12 from contaminants, such as dust particles, by ensuring that the contaminants remain a defined distance away from photomask 12. This may be especially important in a lithography system. During a lithography process, photomask assembly 10 is exposed to electromagnetic energy produced by a radiant energy source within the lithography system. The electromagnetic energy may include light of various wavelengths, such as wavelengths approximately between the I-line and G-line of a Mercury arc lamp, or DUV, VUV or EUV light. In operation, pellicle film 22 is designed to allow a large percentage of the electromagnetic energy to pass through it. Contaminants collected on pellicle film 22 will likely be out of focus at the surface of the wafer being processed and, therefore, the exposed image on the wafer should be clear. Pellicle film 22 formed in accordance with the teachings of the present disclosure may be satisfactorily used with all types of electromagnetic energy and is not limited to lightwaves as described in this application.

Photomask 12 may be formed from a photomask blank using a standard lithography process. The desired pattern may be imaged into a resist layer of the photomask blank using a laser, electron beam or X-ray lithography system. In one embodiment, a laser lithography system uses an Argon-Ion laser that emits light having a wavelength of approximately 364 nanometers (nm). In alternative embodiments, the laser lithography system uses lasers emitting light at wavelengths from approximately 150 nm to approximately 300 nm. In other embodiments, a 25 keV or 50 keV electron beam lithography system uses a lanthanum hexaboride or thermal field emission source. In further embodiments, different electron beam lithography systems may be used. Photomask 12 may be fabricated by developing and etching exposed areas of the resist layer to create a pattern, etching the portions of patterned layer 18 not covered by resist, and removing the undeveloped resist to create patterned layer 18 over substrate 16.

During manufacture and use of photomask 12 and/or pellicle film 22, contaminants may be introduced on the surface such that a haze (e.g., ammonia sulfate or oxalic acid) begins to grow. For example, exposure to electromagnetic energy (e.g., at or below 193 nm lithography) may act as a catalyst for haze growth on the surface of substrate 16 and/or patterned layer 18. A haze may also be formed on the surface substrate 16 and/or patterned layer 18 due to reactions between residue remaining from the manufacturing process and, for example, moisture near the surface.

In order to prevent the haze from growing on the surface, a gas (e.g., nitrogen or an inert gas such as argon or neon) may be introduced proximate to the surface of substrate 16 and/or patterned layer 18. In one embodiment, the gas may be introduced into a storage container that holds one or more photomasks 12 and/or pellicle assemblies 14. The storage container may include a gas source operable to discharge gas into the container. In one embodiment, the gas source may be associated with a lid of the container such that the gas is released into the container as the lid is closed. By storing photomask 12 and/or pellicle assembly 14 in the container including nitrogen or an inert gas, continued haze growth on the surface may be prevented and thus, the properties of photomask 12 and/or pellicle assembly 14 may be maintained. Additionally, the lifetime of photomask 12 and/or pellicle assembly 14 may be increased and/or the amount of cleaning needed to ensure proper operation may be reduced.

In another embodiment, the storage container may be used to hold one or more semiconductor wafers. The gas may prevent a haze from growing on the surface of the semiconductor wafers and therefore, prevent any integrated circuits on the semiconductor wafer from being damaged.

FIGS. 2A and 2B respectively illustrate a top view and a cross-sectional view of an example storage container including a self-contained gas source. As shown in FIG. 2A, gas source 32 may be attached to a back surface of storage container 30. In other embodiments, gas source 32 may be attached to the front or side surfaces of storage container 30. Gas source 32 may be located either inside or outside of storage container 30. If gas source 32 is located outside of storage container 30, a small outlet from gas source 32 may be inserted into a similar size opening in storage container 30 and may provide a path for the gas to be discharged into storage container 30.

Gas source 32 may be a cylinder, cartridge or other similar container that may be used to store a gas. In one embodiment, the gas may be nitrogen. In other embodiments, the gas may be an inert gas. In operation, gas source 32 may be triggered by trigger mechanism 34 to discharge the gas into storage container 30 when lid 36 is closed. Gas source 32 may be used for several opening and closing cycles before being replaced. Gas source 32 may also provide a continuous purge (e.g., positive pressure) for a predetermined period of time until the substrate being stored in storage container 30 may be transferred to another storage container. In other embodiments, gas source 32 may contain a single charge and may be replaced each time storage container 30 is opened.

In one embodiment, storage container 30 may be a compact operable to hold a photomask. The compact may include a lid having a seal (e.g., an O-ring) such that air and other contaminants cannot enter the compact once the lid has been closed. In another embodiment, storage container 30 may be a wafer holder operable to hold one or more semiconductor wafers. In yet another embodiment, storage container 30 may be a stocker operable to hold one or more substrates.

As illustrated in FIG. 2A, storage container 30 may include trigger mechanism 34 such that gas source 32 discharges a gas into storage container 30 when lid 36 is closed. In one embodiment, trigger mechanism 34 may include a spring connected to a rotary valve located on gas source 32. At a certain angle (e.g., less than forty-five degrees) of a hinge connecting lid 36 to the bottom section of storage container 30, the spring may compress and open the rotary valve to allow gas from gas source 32 to flow into storage container 30. As lid 36 closes, the compression of the spring may continue to turn the valve on gas source 32 to the off position. At this point, the gas discharge may be discontinued. In other embodiments, trigger mechanism 34 may be any mechanism that allows gas source 32 to provide a controlled or timed burst of gas.

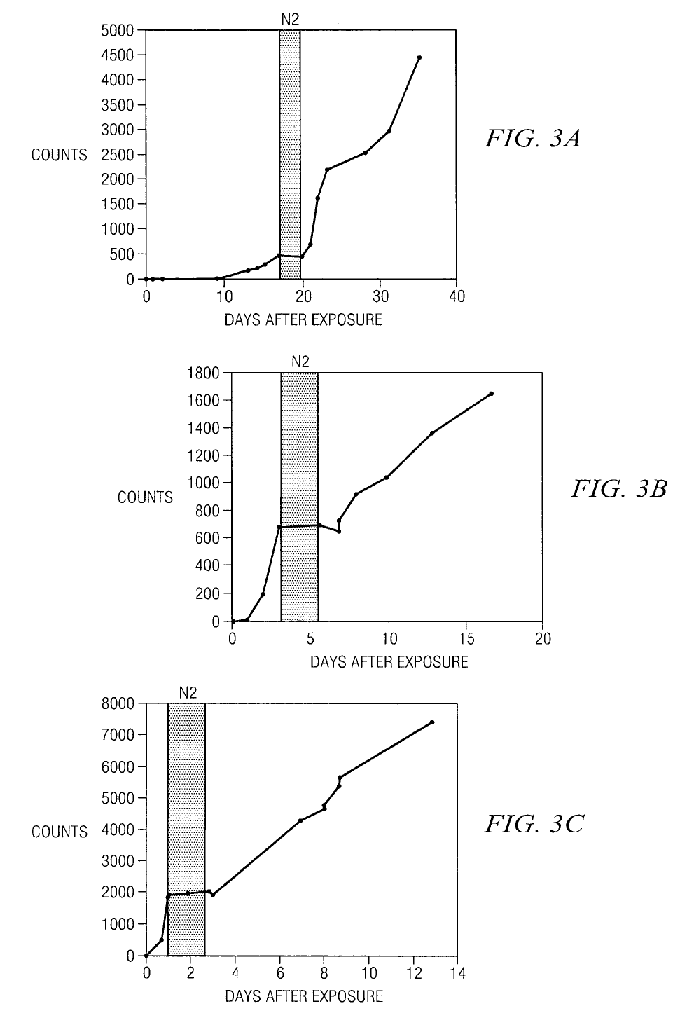

FIG. 3A through 3C illustrate graphs of contaminant growth on a surface of a substrate over time when exposed to a gas according to teachings of the present disclosure. As shown, photomasks already including some haze growth were exposed to nitrogen (N₂) for a certain period of time. In each example, the haze growth stopped during the time that the photomask was exposed to nitrogen independent of the number of days that a haze has been growing on the surface. Additionally, the haze growth continued after the photomask was removed from the nitrogen environment. Therefore, the introduction of nitrogen proximate the surface of a substrate having a layer of haze formed on the surface stops the haze growth while the nitrogen is present.

Although the present disclosure and its advantages have been described in detail, it should be understood that various changes, substitutions and alterations can be made herein without departing from the spirit and scope of the disclosure as defined by the following claims.

What is claimed is:

1. An apparatus for reducing haze growth on a substrate, comprising:
a container operable to store a substrate; and
a gas source coupled to the container, the gas source operable to dispense a gas into the container in order to prevent a haze from growing on a surface of the substrate.

2. The apparatus of claim 1, wherein the container comprises a lid operable to enclose the substrate within the container.

3. The apparatus of claim 2, wherein the gas is dispensed when the lid is closed.

4. The apparatus of claim 1, wherein the gas comprises nitrogen.

5. The apparatus of claim 1, wherein the gas comprises an inert gas.

6. The apparatus of claim 1, wherein the container comprises a compact or a stocker operable to hold a photomask.

7. The apparatus of claim 1, wherein:
the container is operable to store a plurality of substrates; and
the gas source is operable to prevent haze growth on the surface of the plurality of substrates.

8. The apparatus of claim 1, wherein the substrate comprises a photomask substrate.

9. The apparatus of claim 1, wherein the substrate comprises a semiconductor wafer.

10. The apparatus of claim 1, wherein the substrate comprises a pellicle film.

11. A method for reducing haze growth on a substrate, comprising:
providing a container operable to hold a substrate, the container operable to enclose the substrate within the container;
providing a gas source coupled to the container; and
dispensing a gas from the gas source, the gas operable to prevent a haze from growing on the substrate.

12. The method of claim 11, wherein the container comprises a lid operable to enclose the substrate within the container.

13. The method of claim 12, further comprising:
activating a trigger mechanism as the lid is being closed; and
dispensing the gas from the gas source when the lid is closed.

14. The method of claim 13, wherein the trigger mechanism comprises a rotary valve coupled to a spring such that compression of the spring controls the discharge of the gas source.

15. The method of claim 11, wherein the gas comprises nitrogen.

16. The method of claim 11, wherein the gas comprises an inert gas.

17. The method of claim 11, wherein the container comprises a compact or a stocker operable to hold a photomask.

18. The method of claim 11, wherein:
the container is operable to hold a plurality of substrates; and
the gas source is operable to prevent haze growth on the surfaces of the plurality of substrates.

19. The method of claim 11, wherein the substrate comprises a photomask substrate.

20. The method of claim 11, wherein the substrate comprises a semiconductor wafer.

21. The method of claim 11, wherein the substrate comprises a pellicle film.
